(19) United States
(12) Patent Application Publication
(10) Pub. No.: US 2013/0317295 A1
(43) Pub. Date: Nov. 28, 2013

(54) Light assembly for remote visual inspection apparatus

(71) Applicant: Bradford Morse, Syracuse, NY (US)

(72) Inventor: Bradford Morse, Syracuse, NY (US)

(73) Assignee: GE Inspection Technologies, Schenectady, NY (US)

(21) Appl. No.: 11/647,680

(22) Filed: Dec. 29, 2006

(51) Int. Cl. A61B 1/06; A61B 1/07; A61B 1/05

(52) U.S. Cl. CPC A61B 1/0653; A61B 1/05; A61B 1/07
USPC 600/109

(57) ABSTRACT

An apparatus for use as a light assembly in remote visual inspection devices is provided. The light assembly may consist of a laser diode coupled to a fiber optic bundle that transmits collimated laser light onto a wavelength converter located in the distal end of the remote video inspection system. Wavelength converters consisting of phosphorescent materials can be used to convert collimated laser light into white light for inspection illumination purposes.

### FIELD OF THE INVENTION

This invention relates generally to remote visual inspection devices, and, in particular, to a light assembly for use in remote visual inspection devices.

### BACKGROUND OF THE INVENTION

Inspection units for remotely inspecting the interior portions of a body cavity of a living thing, such as a person, for medical diagnosis or a medical procedure, or for inspection or possibly repair of interior portions of industrial equipment, such as a boiler, a pipe or an engine, are known: Such devices commonly employ inefficient high intensity discharge lamps, e.g. xenon, high pressure mercury, or metal halide lamps that generate a significant amount of heat and are susceptible to breaking, rupture and sudden failure. High intensity discharge lamps are incapable of readily being turned on and off as needed by an inspector, further increasing their inefficiency and impairing their utility.

One approach used to resolve the deficiencies in utilizing high intensity discharge lamps has been the placement of low power light emitting diodes in the distal end of the remote visual inspection device to provide target illumination. However, using light emitting diodes in the distal end of the device has also been problematic in that such an arrangement produces undesired heat within both the distal end of the remote visual inspection device, and the target area on which the inspection is taking place. In addition, such arrangements typically require multiple LEDs in order to provide sufficient and evenly dispersed lighting on a given target. The use of multiple LEDs requires a significant amount of physical space and limits the ability to miniaturize inspection devices for insertion into small, confined spaces. Further limitations occur as multiple inefficient LEDs reduce the gains that LEDs can provide.

Another approach used to provide illumination using LEDs involves placement of the LED in a proximal location within the remote video inspection device and using a fiber optic connection to channel the emitted light to the distal end of the device. However, placement of the LED in a proximal position also results in inefficiencies because of the inherent loss associated with focusing the broad angular emission of an LED onto an optical fiber.

With either high intensity discharge lamps or LEDs, the light emitted from the distal end of the remote visual inspection device requires additional optics to diffuse the light to match the field of view or direction of the imager optics. To accomplish this, additional illumination optics are used that reduce the light that illuminates the target under inspection.

These systems, as described, have deficiencies in aspects such as efficiency, portability and convenience that are general needs in the industry. There is a need for borescope and endoscope systems that provide improved convenience for the user while offering greater illumination of target, image quality, technical capabilities, better maintainability, and more favorable economics.

### BRIEF DESCRIPTION OF THE DRAWINGS

The features, aspects and advantages of the invention can be better understood with reference to the drawings, described below, and the claims. The drawings are not necessarily to scale, emphasis instead generally being placed upon illustrating the principles of the invention. In the drawings, like numerals are used to indicate like parts throughout the various views.

FIG. 1a shows an exemplary remote visual inspection apparatus, having elements of an illumination system highlighted;

FIG. 1b shows and exemplary camera for use in a remote visual inspection apparatus;

FIG. 2 shows an exemplary distal end of a remote visual inspection apparatus with tip removed from the camera;

FIG. 3 shows an exemplary distal end of a remote visual inspection apparatus with tip attached to the camera;

FIGS. 4-6 depict the distal end of a remote visual inspection apparatus with various tip attachments;

FIGS. 7-9 depict exemplary viewing and illumination characteristics of a remote visual inspection apparatus made possible through the use of various tip attachments;

FIG. 10 is a block electrical layout diagram of a remote visual inspection apparatus electrical and control system;

FIG. 11 is a cross sectional view of an elongated inspection module of a remote visual inspection apparatus.

FIG. 12 shows an exemplary distal end of a remote visual inspection apparatus with tip removed from the camera.

### DETAILED DESCRIPTION OF THE INVENTION

As discussed in detail below, there is provided in one embodiment a low power illumination system comprising a light source directing light onto a fiber optic bundle that carries light to a distal section having an optical diffuser. The light source can be a laser diode assembly. Use of the laser diode can reduce power consumption to between 1 and 3 Watts, as opposed to 24 to 75 Watts when using a high intensity discharge lamp. Reduced power consumption results, in turn, in lower heat generation and increased efficiency of the remote visual inspection apparatus. In addition, use of a laser diode provides an inspector with wide latitude to turn the illumination source of the remote visual inspection apparatus on and off as a given inspection situation requires, thereby increasing the overall efficiency of the device. In one embodiment, the optical diffuser can be provided by a wavelength converter which, in addition to diffusing light, converts the wavelength of received light from a narrow wavelength band into white light to facilitate color imaging.

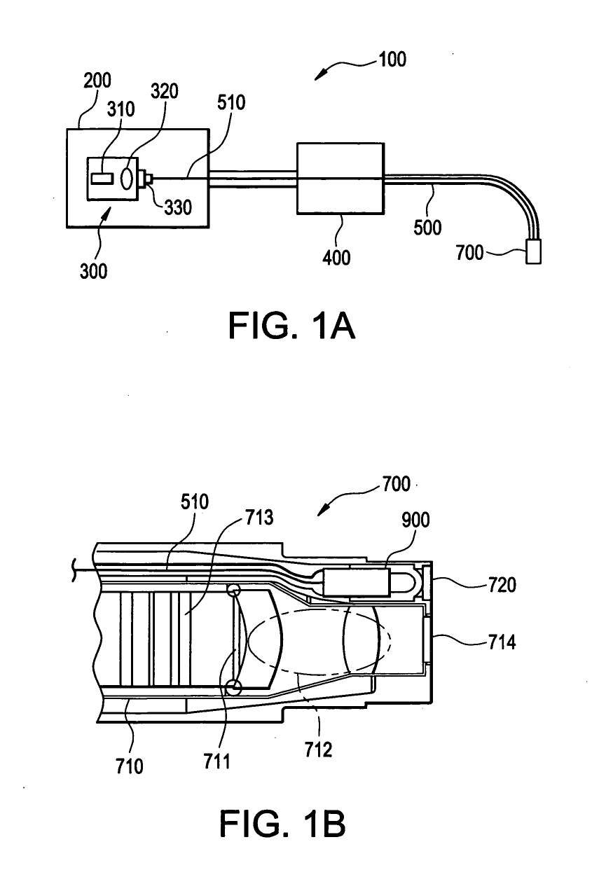

FIG. 1 a shows an exemplary remote visual inspection apparatus 100 having elements of an illumination system highlighted. In one embodiment, a laser diode assembly 300 may be located within a base unit 200. The laser diode assembly 300 can also be located outside of the base unit, for example in the control and display device 400, elongated inspection module 500, camera 700, or detachable tip 800 described below. The laser diode assembly 300 may include a laser diode 310 that emits narrow band light through a collimating lens 320. Collimating lens 320 captures diffuse light rays emanating from the laser diode 310 and focuses them onto the proximal end of a fiber optic bundle 510. This fiber optic bundle 510 may be selected based on the wavelength to be emitted or converted at the distal end of camera 700. Arrangement of the elements of the laser diode assembly 300 according to FIG. 1a reduces light loss due to scattering and maximizes the intensity of light incident on the fiber optic bundle 510. The fiber optic bundle 510 may consist of one or more optical fibers. The fiber optic bundle 510 may be connected to the laser diode assembly 300 by a bundle connector 330. Bundle connector 330 can be clamped around the distal end of fiber optic bundle 510 and coupled with interlocking components on the laser diode assembly 300 such that, when connected, the distal end face of the fiber optic bundle 510 is located at the approximate focal point of collimating lens 320.

The fiber optic bundle 510 can extend outward from the laser diode assembly 300 to the distal end of a camera 700, which may be integrated into to the distal end of the elongated inspection module 500. The control and display device 400 provides an inspector with the ability to control the functions and operating parameters of the remote visual inspection apparatus 100, and to display imaging results, operational metrics and other information. The fiber optic bundle 510 may be fully encapsulated by the control and display device 400 and the elongated inspection module 500. As shown in FIG. 1b, Camera 700 may include a camera canister 710 which houses a camera imaging window 714, a camera lens system 712, a two dimensional solid state image sensor 711, and image processing circuitry 713. The image sensor 711 can be, e.g., a charge coupled device (CCD) or CMOS image sensor, and can include a plurality of pixels formed in a plurality of rows and columns. The image sensor 711 can be provided on an integrated circuit, and can generate image signals in the form of analog voltages representative of light incident on each pixel of the image sensor. The fiber optic bundle 510 can run between the outside of the camera canister 710 and the outer wall of the elongated inspection module 500 such that it does not impact the field of view or operation of the camera 700. Attached to the distal end of the fiber optic bundle 510 may be an optical diffuser 900 that scatters the collimated light emitted by the distal end of the fiber optic bundle 510. The optical diffuser 900 may consist of an optically clear material that may be deposited at the distal end of the fiber optic bundle 510, in which the distal surface of the material may include geometrically faceted surface features. Light emanating from the distal end of the fiber optic bundle 510 passes through the clear optical diffuser 900 and, prior to exiting the distal end of the diffuser, passes through the surface facets where it can be skewed at various angles depending on the geometry of the given facet it has passed through. Use of the optical diffuser 900 acts to disperse the collimated light and increase the overall radius of illumination. The optical diffuser may be positioned parallel and adjacent to the camera canister 710, and disposed at the distal end of the camera 700. The shape of the optical diffuser 900 may be varied in order to fit securely within the camera 700 without interfering with the operation or orientation of other components. A sealed camera illumination window 720 is positioned at the distal end of the optical diffuser to allow light to emanate from the distal face of the camera 700. The camera illumination window 720 is comprised of a clear material that allows light to pass through it with little or no optical effect, e.g. a piece of flat glass, and can be sealed to prevent liquids and other materials from entering the camera 700.

In another embodiment, the optical diffuser 900 may consist of a wavelength converter. The wavelength converter may be a material, such as a phosphor or quantum dots, that, when stimulated by narrow band laser light, is energized to an elevated energy state causing that material to fluoresce and emit white light. The optical diffuser 900 including a wavelength converter may consist of a phosphor coated clear material, e.g. a phosphor coated glass that is deposited at the distal end of the fiber optic bundle 510. As light from the laser diode 310 strikes the wavelength converter, the incident photons excite the molecules of the wavelength converter, raising them to an elevated energy state. With sufficient excitation, the wavelength converter begins to fluoresce and reemit the absorbed energy in the form of white light. The white light emitted by the wavelength converter is inherently non-collimated and well suited for target illumination, thereby eliminating the need for any secondary optics to diffuse the laser light. In addition, little heat is generated by the light emission from the optical diffuser, preventing heat related damage or distortion effects from occurring to both the camera 700 and the inspection target. A device suitable for an embodiment utilizing a wavelength converter that includes the laser diode assembly 300, fiber optic bundle 510, and the optical diffuser 900 is the NDAW001 high luminance light source available from Nichia Corporation of Tokushima, Japan. The color temperature of the light emitted can be altered through choice of laser diode and phosphor composition.

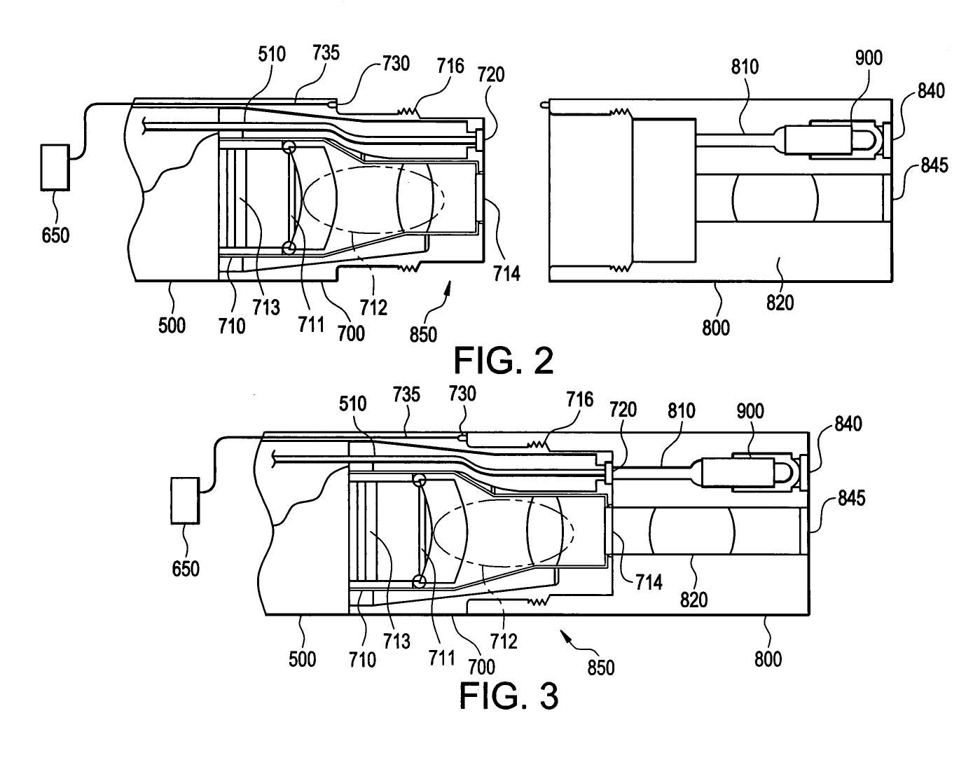

FIG. 2 shows one embodiment in which a camera head assembly 850 is located at the distal end of the elongated inspection module 500. The camera head assembly 850 can be disintegrated into multiple components, including the camera 700 and a detachable tip 800. Use of detachable tips 800 allows an inspector to modify the optical and illumination characteristics of the remote visual inspection apparatus 100 of FIG. 1 a by placing additional optical and illumination components in front of the camera 700 to obtain a desired effect. Detachable tip 800 may include a tip lens system 820 that provides preliminary optical manipulation of an inspection target image entering through the tip imaging window 845, prior to that image entering the camera 700. Selection of different optical lenses or combinations of lenses can be made in order to obtain a desired optical effect.

FIG. 3 shows one embodiment in which the detachable tip 800 can be attached to the camera 700 by screwing the tip 800 onto a set of tip threads 716 located on the distal end of the camera 700. In one embodiment, the optical diffuser 900 can be located within the detachable tip 800. The detachable tip 800 may include a tip waveguide 810 that provides a path for light emanating from the distal end of the fiber optic bundle 510 and incident on the proximal face of the attached tip 800 to travel to the optical diffuser 900 located somewhere within the detachable tip 800. The tip waveguide 810 and optical diffuser 900 can be arranged within the tip 800 such that they do not interfere with the imaging optics or desired optical effect of the tip 800. Light emitted by the optical diffuser 900 travels through the tip illumination window 840 to allow light to emanate from the detachable tip 800. The tip illumination window 840 is comprised of a clear material that allows light to pass through it with little or no optical effect, e.g. a piece of flat glass, and can be sealed to prevent liquids and other materials from entering the detachable tip 800. Detachable tip 800 may include an optical diffuser 900 that also acts as a wavelength converter, e.g. a phosphor or quantum dots. Narrow band laser light is emitted from the fiber optic bundle 510 located in the distal end of the camera 700, travels into the detachable tip 800 along the optical waveguide 810, and is incident on the optical diffuser 900. The incident photons excite the molecules of the wavelength converter, raising them to an elevated energy state. With sufficient excitation, the wavelength converter begins to fluoresce and reemit the absorbed energy in the form of white light, which is then emitted from the tip 800 onto the inspection target.

With reference to FIG. 2, when the detachable tip 800 is removed, light emitted from the distal face of the camera 700 remains unconverted narrow band laser light. The narrow band laser light emitted by the laser diode can be of an intensity and wavelength as to cause serious injury if handled inappropriately. In order to ensure safe operation of the remote visual inspection apparatus, in one embodiment, a safety switch 730 may be located at the distal end of the camera to turn off the laser diode when a tip 800 is not attached. Camera head assembly 850 can be adapted so that safety switch 730 can be in communications with a processor 650 by an electrical connection 735, and can be activated by the presence of an optical tip 800, allowing electrical power to flow to the laser diode. Camera head assembly 850 can be adapted so that when the optical tip 800 is removed, the electrical connection is broken and the laser diode is automatically turned off.

While it may be useful to incorporate safety switch 730 in some embodiments, it may be useful in other embodiments to disable the safety switch 730, or to provide a remote visual inspection device without a safety switch 730. These embodiments allow the remote visual inspection device to be operated without the detachable tip 800 being affixed to the camera 700. The light emitted from the distal face of the camera remains unconverted narrow band laser light, and can be utilized by an inspector for purposes requiring narrow band light inspection, e.g. ultra violet and infra red. One such inspection is fluorescent penetrant for non-destructive testing. In another embodiment, a detachable tip 800 including an optical diffuser 900 that does not include a wavelength converter can be used. In a configuration that lacks a wavelength converter, the light emitted from the distal face of the tip 800 remains unconverted narrow band laser light, and can be utilized by an inspector for purposes requiring narrow band light inspection, e.g. ultra violet and infra red. Safety switch 730 is activated by the presence of the narrow band tip, and power is supplied to the laser diode. However, being that an inspector has specifically selected and attached the narrow band tip for use, the safety concerns associated with inadvertent misuse of the remote visual inspection apparatus while emitting narrow band light are mitigated.

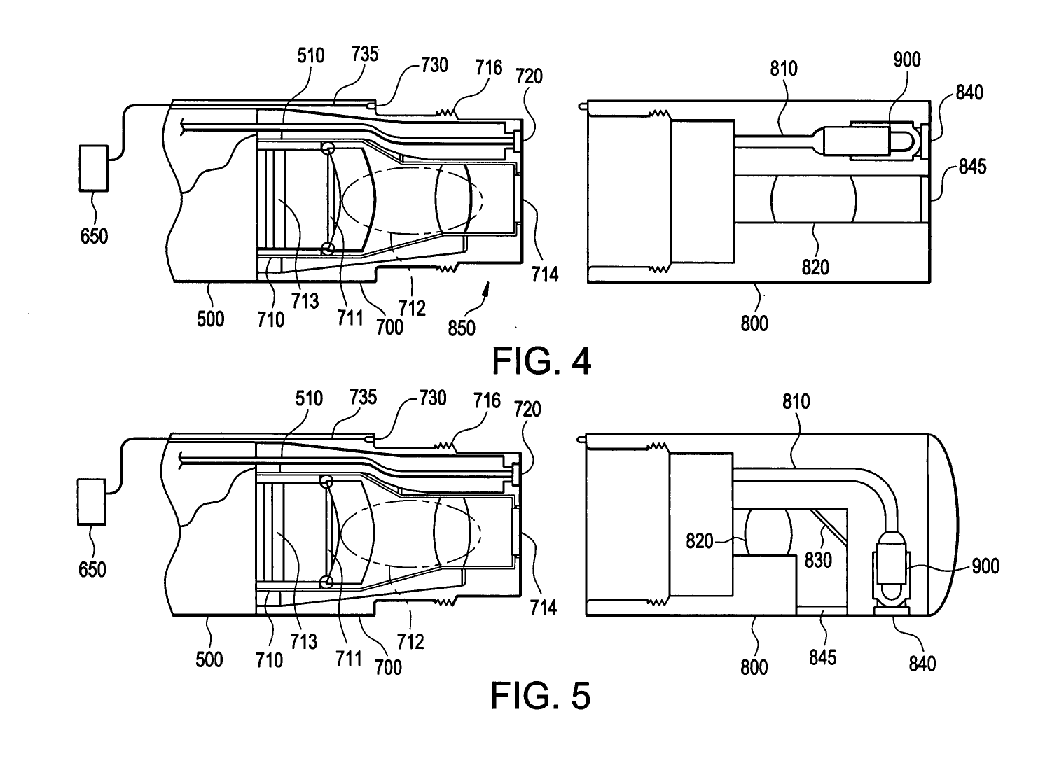

With reference to FIGS. 4-6, in one embodiment, the remote visual inspection device can be provided in a kit, wherein the apparatus is adapted so that more than one detachable tip 800 can be provided. Each tip 800 provided may include different optical and illumination characteristics, controlled through different combinations and configurations of the tip waveguide 810, the tip lens system 820, the tip mirror 830, and the optical diffuser 900. Differently shaped tip waveguides 810 can be used to direct light from the distal end of the camera 700 to different locations within the tip 800 from which the light is emitted. Similarly, the optical diffuser 900 can be shaped and located in various locations throughout the tip 800 in order to create a customized lighting effect for the use and field of view associated with a given tip lens system 820. As shown in FIGS. 4-6, the optical waveguide 810 and the optical diffuser 900 can be shaped in order to direct the emitted white light in the desired direction and angle of viewing. This provides significant advantage as differently shaped waveguides eliminates the need for multiple illumination optics that reduce the efficiency of the light.

In one exemplary embodiment, shown in FIG. 4, the detachable tip 800 may provide forward viewing and illumination of an inspection target. In this embodiment, tip waveguide 810 extends from the proximal end of the detachable tip 800 to the optical diffuser 900 located in the distal end of the detachable tip 800. Tip illumination window 840 is located on the distal face of the detachable tip 800, thereby providing forward illumination of an inspection target. Similarly, the tip lens system 820 is aligned approximately parallel to the tip waveguide 810, with the tip imaging window 845 located on the distal face of the detachable tip 800 such that forward viewing can be achieved.

In another exemplary embodiment, shown in FIG. 5, the detachable tip 800 may provide side viewing and illumination of an inspection target. In this embodiment, tip waveguide 810 can extend from the proximal end of the detachable tip 800 to the optical diffuser 900 located near a sidewall of the detachable tip 800. Tip illumination window 840 can also be located on the sidewall of the detachable tip 800, thereby providing side illumination of an inspection target. In this embodiment, the tip waveguide 810 and optical diffuser 900 are configured so as to provide a small, focused radius of illumination. In order to provide side viewing of an inspection target, the tip imaging window 845 can also be located on the sidewall of the detachable tip 800. Tip mirror 830 is provided to redirect light entering the tip imaging window 845 back towards the tip lens assembly 820 and the proximal end of the detachable tip 800. The light passes through the tip lens system 820, through the proximal end of the detachable tip 800, and is incident on the camera imaging window 714.

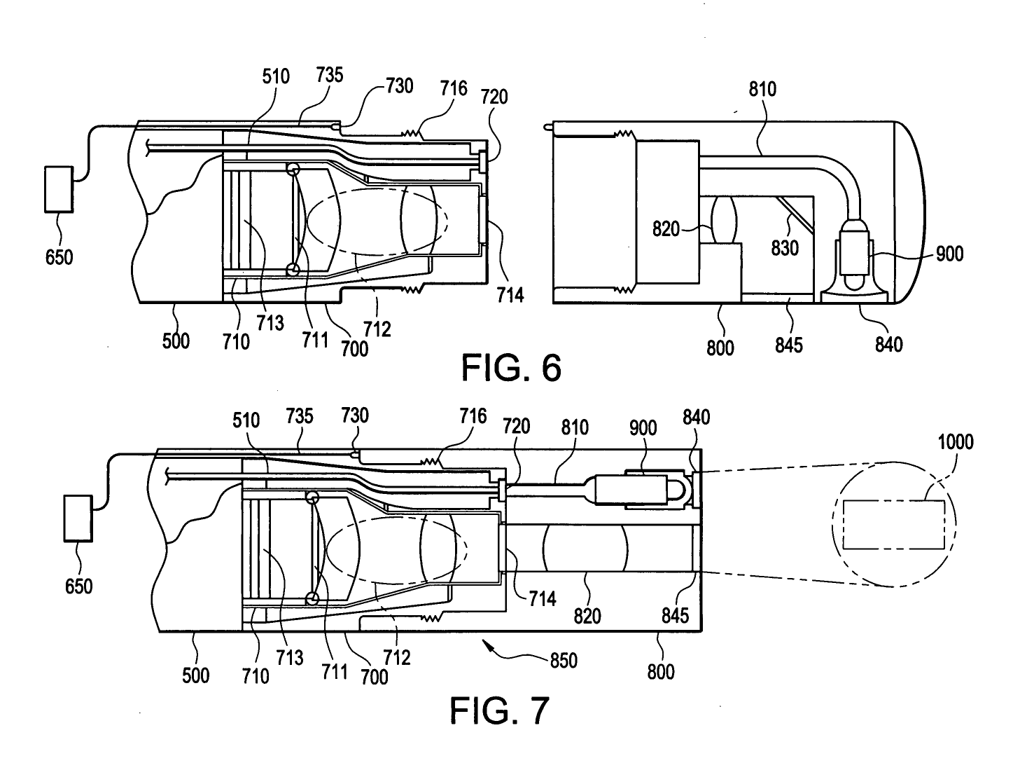

In another exemplary embodiment, shown in FIG. 6, the detachable tip 800 may provide wide angle side viewing and illumination of an inspection target. In this embodiment, tip waveguide 810 can extend from the proximal end of the detachable tip 800 to the optical diffuser 900 located near a sidewall of the detachable tip 800. Tip illumination window 840 can also be located on the sidewall of the detachable tip 800, thereby providing side illumination of an inspection target. In this embodiment, the tip waveguide 810 and optical diffuser 900 are configured so as to provide a large radius of illumination. In order to provide side viewing of an inspection target, the tip imaging window 845 can also be located on the sidewall of the detachable tip 800. Tip mirror 830 is provided to redirect light entering the tip image window 845 back towards the tip lens assembly 820 and the proximal end of the detachable tip 800. The light passes through the tip lens system 820, through the proximal end of the detachable tip 800, and is incident on the camera imaging window 714. In this embodiment, the tip lens system 820 is such that it provides for a wide angle field of view and enlarged viewing radius.

FIGS. 7-9 show an exemplary view of the lighting and viewing effects that can be obtained when different tips 800 included within a kit are attached to the camera 700. In one exemplary embodiment, shown in FIG. 7, detachable tip 800 may provide forward viewing and illumination of an inspection target 1000. When the detachable tip 800 is attached to the camera 700 to form the camera head assembly 850, light waves travel from the fiber optic bundle 510 through the camera illumination window 720 into the tip waveguide 810, and are incident on the optical diffuser 900 which fluoresces white light out of the tip illumination window 840 . Light reflected off of the inspection target 1000 enters the detachable tip 800 though the tip imaging window 845 and passes through to the tip lens system 820. The light then passes through the proximal end of the detachable tip 800 and into the camera imaging window 714.

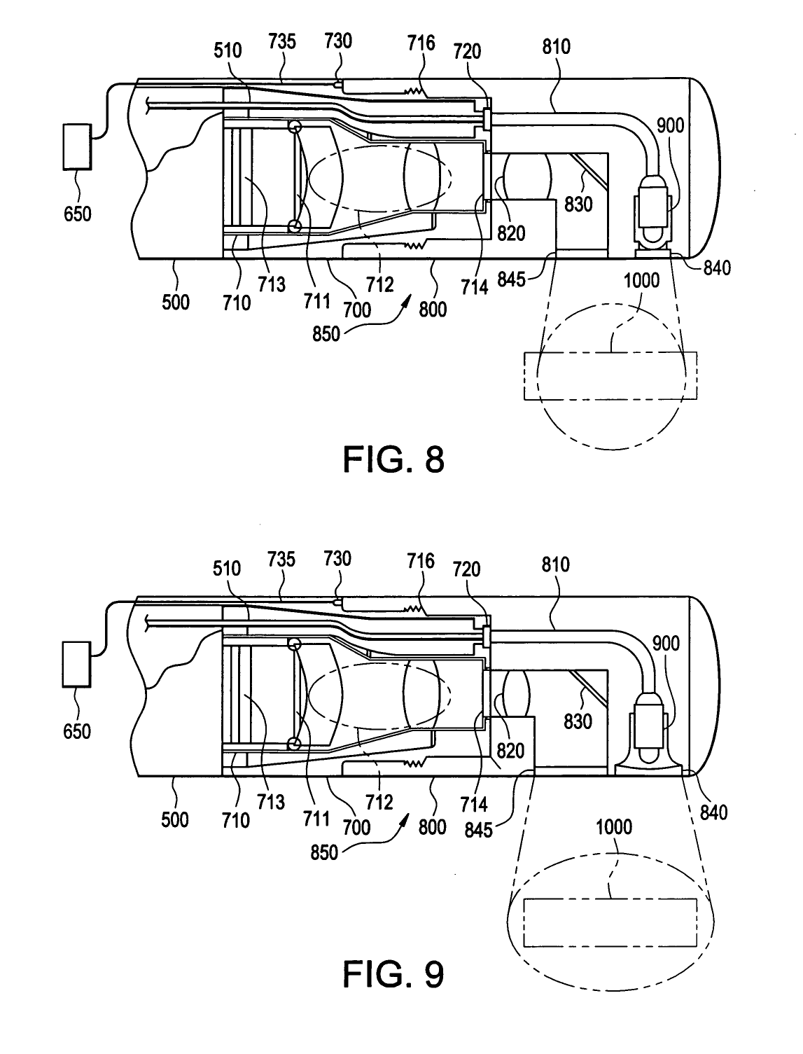

In another exemplary embodiment, shown in FIG. 8, detachable tip 800 may provide narrow field side viewing and illumination of an inspection target 1000. When the detachable tip 800 is attached to the camera 700 to form the camera head assembly 850, light waves travel from the fiber optic bundle 510 through the camera illumination window 720 into the tip waveguide 810, and are guided to the optical diffuser 900 which fluoresces white light out of the tip illumination window 840 located in the sidewall of the tip 800. In this embodiment, the tip waveguide 810 and optical diffuser 900 are configured so as to provide a narrow radius of illumination. Light reflected off of the inspection target 1000 enters the detachable tip 800 though the tip imaging window 845, and is reflected towards the proximal end of the detachable tip 800 by tip mirror 830. The light then passes through the tip lens system 820, through the proximal end of the detachable tip 800, and into the camera imaging window 714. In this embodiment, the tip lens system 820 is such that it provides for a narrow field of view.

In another exemplary embodiment, shown in FIG. 9, detachable tip 800 may provide wide angle viewing and illumination of an inspection target 1000. When the detachable tip 800 is attached to the camera 700 to form the camera head assembly 850, light waves travel from the fiber optic bundle 510 through the camera illumination window 720 into the tip waveguide 810, and are guided to the optical diffuser 900 which fluoresces white light out of the tip illumination window 840 located in the sidewall of the tip. In this embodiment, the tip waveguide 810 and optical diffuser 900 are configured so as to provide a broad radius of illumination. Light reflected off of the inspection target 1000 enters the detachable tip 800 though the tip imaging window 845 and is reflected towards the proximal end of the detachable tip 800 by tip mirror 830. The light then passes through the tip lens system 820, through the proximal end of the detachable tip 800, and into the camera imaging window 714. In this embodiment, the tip lens system 820 is such that it provides for a wide angle field of view and enlarged viewing radius.

Repeated use of tips 800 including a phosphor based wavelength converter may result in diminished white light emission intensity over time. Use of interchangeable tips 800 allows for replacement of optical diffusers including wavelength converters in order to maintain the optimal lighting efficiency of a given remote visual inspection apparatus or to allow for use of continually improving wavelength converter technologies.

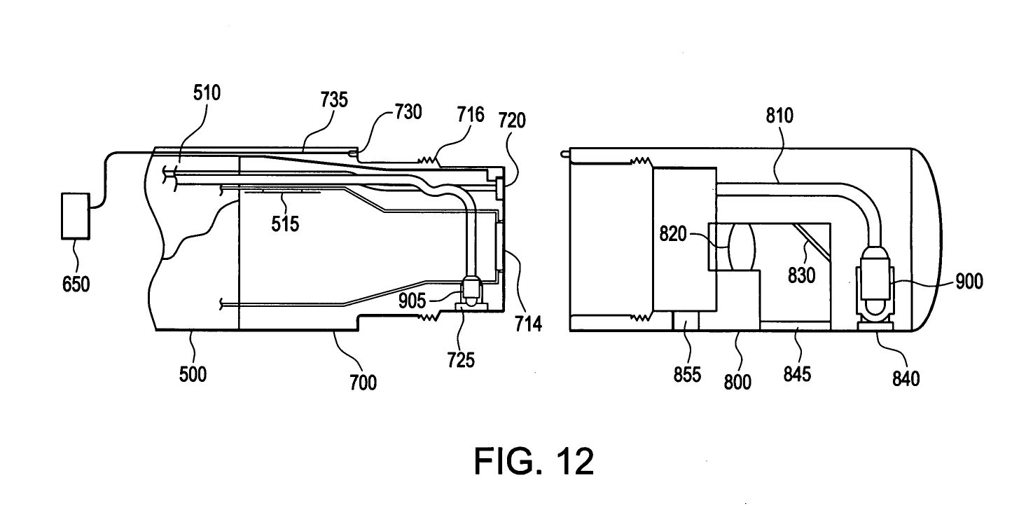

In another exemplary embodiment, shown in FIG. 12, side illumination of an inspection target may be provided with or without the use of a tip waveguide 810 in the detachable tip. In this embodiment as in the embodiment shown in FIG. 5 and discussed above, tip waveguide 810 can extend from the proximal end of the detachable tip 800 to the optical diffuser 900 located near a sidewall of the detachable tip 800. Tip illumination window 840 can also be located on the sidewall of the detachable tip 800, thereby providing side illumination of an inspection target. In addition or in lieu of the use of a tip waveguide 810 to provide side illumination, the exemplary embodiment shown in FIG. 12 can provide side illumination using a fiber optic bundle 515 in the camera 700 and attaching the distal end of the bundle 515 to an optical diffuser 905 adjacent to a sealed camera illumination window 725 to allow light to emanate from the side face of the camera 700. The fiber optic bundle 515 used for side illumination from the camera 700 can also be part of the fiber optic bundle 510 used for “straight” illumination from the camera as shown in FIGS. 4-6. In order to accommodate this side illumination from the camera, detachable tip 800 has a tip illumination window 855 positioned to allow light from the optical diffuser 905 to emanate from the side face of the camera 700 and detachable tip 800. In minimizing the optics necessary to accomplish this side illumination, reduction of light that illuminates the target under inspection is minimized.

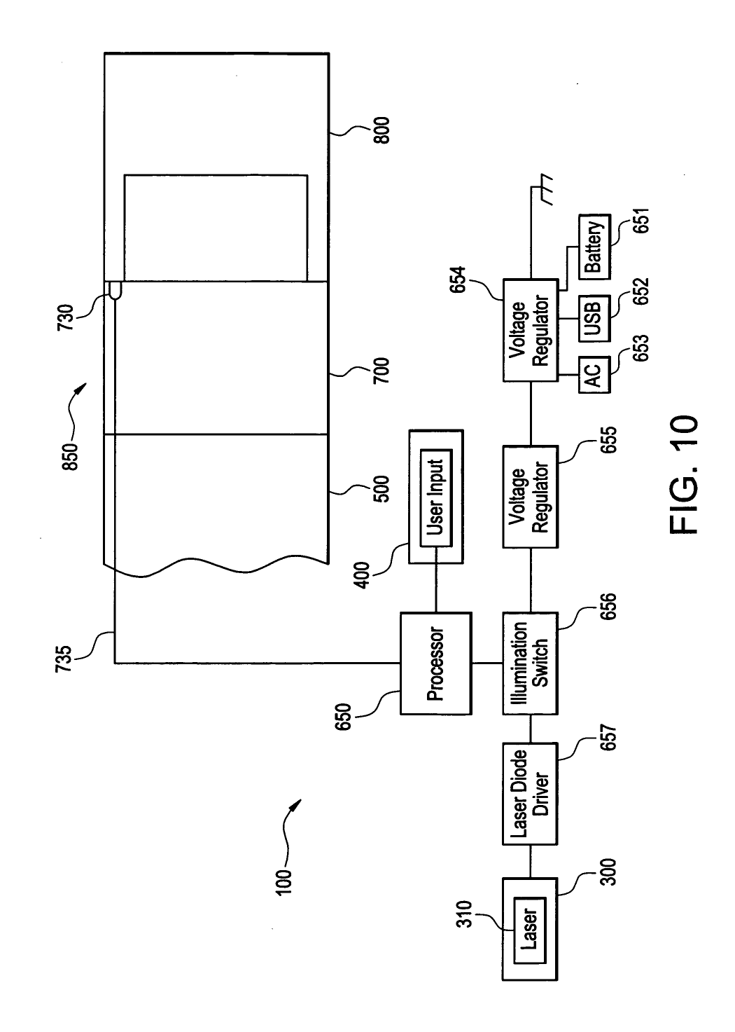

FIG. 10 shows an exemplary block electrical layout diagram for the illumination system of a remote visual inspection apparatus 100 with laser diode assembly 300. In one embodiment, power may be supplied to the system through a power supply 654, which can accept as electrical sources an alternating current source 653, a USB source 652, or a direct current battery source 651. Power from the power supply 654 may then be connected to a voltage regulator 655 that can modify the output of the power supply in order to achieve the voltage required by the illumination system. The power output from the voltage regulator 655 may be connected to an illumination switch 656. When the tip 800 is attached to the camera 700 forming the camera head assembly 850, the safety switch 730 is closed. This completes an electrical connection 735 between a processor 650 and the safety switch 730. The processor 650 can be programmed such that when the safety switch 730 is open, the processor 650 opens the illumination switch 656. Leaving the illumination switch 656 open breaks the electrical connection between the voltage regulator 655 and the rest of the illumination system, thereby preventing power from reaching the laser diode 310 and turning the laser diode 310 off. The processor can also be programmed such that when the safety switch 730 is closed, the processor 650 closes the illumination switch 656. When the illumination switch 656 is closed, power flows from the voltage regulator 655 to a laser diode driver 657. The laser diode driver 657 monitors the operation and output of the laser diode 310 and controls the power delivered to the laser diode 310 in order to maintain uniform operation. Therefore, when the illumination switch 656 is closed, power flows from the voltage regulator 655 through the illumination switch 656, to the laser diode driver 657, and eventually to the laser diode 310 causing it to operate and emit laser light. Alternatively, the processor can be instructed by an inspector, using control and display device 400, to override the safety switch 730 and close or open the illumination switch, thereby turning the illumination system on and off as desired by that inspector.

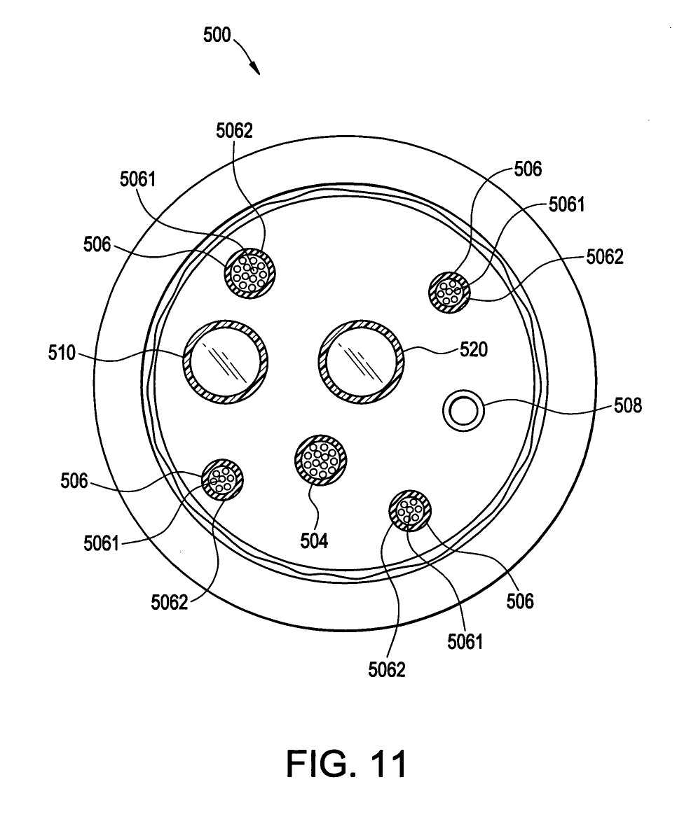

FIG. 11 shows an exemplary cross sectional view of the elongated inspection module 500. The elongated inspection module 500 carries fiber optic bundles 510, cable wiring bundle 504 (including flexible electrical conductors), articulation cable assemblies 506, and a working channel 508. The articulation assemblies 506 provide for bending of the elongated inspection module at its distal end. The articulation assemblies can be provided by a stranded cable 5061 encased by an outer spring conduit 5062. The working channel 508 allows manipulation of a tool (e.g. a hook, a brush, or a magnet) extending from camera head assembly 850.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### ELEMENT LIST

100 Remote viewing apparatus
200 Base unit
300 Laser diode assembly
310 Laser diode
320 Collimating lens
330 Bundle connector
400 Control and display
500 Elongated inspection module
504 Cable wiring bundle
506 Articulation cable assembly
5061 Stranded cable
5062 Outer spring conduit
508 Working channel
510 Fiber optic bundle
515 Fiber optic bundle
650 Processor
651 Battery source
652 USB source
653 AC source
654 Power supply
655 Voltage regulator
656 Illumination switch
657 Laser driver
700 Camera
710 Camera canister
711 Image sensor
712 Camera lens system
713 Imaging circuitry
714 Camera imaging window
716 Tip threads
720 Camera illumination window
725 Camera illumination window
730 Safety switch
735 Electrical Connection
800 Detachable Tip
810 Tip waveguide
820 Tip lens system
830 Tip mirror
840 Tip illumination window
845 Tip imaging window
850 Camera head assembly
855 Tip illumination window
900 Optical diffuser
905 Optical diffuser
1000 Inspection Target

1. A remote visual inspection apparatus comprising:
an elongated inspection module;
a camera head assembly disposed at the distal end of said elongated inspection module, the camera head assembly comprising an electric circuit, a detachable tip, and a safety switch, the safety switch for opening the electric circuit when the detachable tip is detached from the camera head assembly;
a light housing including a light source;
a laser diode assembly for emitting narrow band laser light disposed in said light housing, the laser diode assembly coupled to the electric circuit such that it automatically turns off when the detachable tip is detached from the camera head assembly;
a fiber optic bundle having a proximal end and a distal end, the proximal end being disposed in said light housing for receiving the narrow band laser light from said laser diode assembly, said fiber optic bundle extending through said elongated inspection module; and
an optical diffuser attached to said distal end of said fiber optic bundle and disposed only in said camera head assembly, said optical diffuser for receiving the narrow band laser light emitted from said distal end of said fiber optic bundle to convert said narrow band laser light to white light.

2. The remote visual inspection apparatus of claim 1, wherein said optical diffuser comprises a phosphor that is energized by the narrow band laser light and fluoresces to emit the white light.

3. (canceled)

4. The remote visual inspection apparatus of claim 1, wherein said optical diffuser is disposed in said detachable tip.

5. The remote visual inspection apparatus of claim 1, wherein said light housing is provided by a base unit.

6. The remote visual inspection apparatus of claim 1, wherein said light housing is provided by a control and display module.

7. A remote visual inspection apparatus comprising:
a control and display module;
an elongated inspection module extending from said control and display module, said elongated inspection module being such that it can be configured to be articulable;
a camera head assembly comprising a camera and a detachable tip both disposed at the distal end of said elongated inspection module, said camera head assembly including a two dimensional image sensor and an imaging lens in the camera for focusing an image onto said two dimensional image sensor;
a fiber optic bundle encapsulated by said elongated inspection module;
a laser diode assembly for emitting narrow band laser light, wherein said fiber optic bundle is disposed so that a proximal end of said fiber optic bundle receives said narrow band laser light from said laser diode assembly, conducts said narrow band laser light through said elongated inspection module, and emits said narrow band laser light from its distal end at the camera head assembly;
a first wavelength converter disposed only in said camera head assembly for receiving a first portion of said narrow band laser light emitted from the distal end of a portion of said fiber optic bundle, said wavelength converter for converting the received first portion of the narrow band laser light into a first white light so that a target area is illuminated by the first white light; and
a second wavelength converter disposed only in said camera head assembly for receiving a second portion of said narrow band laser light emitted from the distal end of a second portion of said fiber optic bundle, said second wavelength converter for converting the received second portion of the narrow band laser light into a second white light.

8-9. (canceled)

10. The remote visual inspection apparatus of claim 7, wherein said second wavelength converter is disposed in said detachable tip.

11-15. (canceled)

16. A remote visual inspection apparatus comprising:
an elongated inspection module;
a camera head assembly disposed at the distal end of the elongated inspection module, the camera head assembly comprising an electric circuit, a detachable tip, and a safety switch, the safety switch for opening the electric circuit when the detachable tip is detached from the camera head assembly;
a light housing including a laser diode for emitting narrow band laser light, the laser diode coupled to the electric circuit such that it automatically turns off when the detachable tip is detached from the camera head assembly;
a fiber optic bundle comprising a plurality of optical fibers, the fiber optic bundle having a proximal end and a distal end, the proximal end disposed in the light housing for receiving the narrow band laser light from the laser diode, the fiber optic bundle extending through the elongated inspection module for emitting the narrow band laser light at the camera head assembly; and
a first an optical diffuser disposed only in the camera head assembly, the first optical diffuser for receiving the emitted narrow band laser light and for converting the narrow band laser light to white light.

17. The remote visual inspection apparatus of claim 16, wherein the optical diffuser is provided by a wavelength converter.

18. The remote visual inspection apparatus of claim 16, wherein the camera head assembly comprises a camera and the detachable tip.

19. The remote visual inspection apparatus of claim 18, wherein the first optical diffuser is disposed in the camera.

20. The remote visual inspection apparatus of claim 16, wherein the light housing is provided by a base unit.

21. The remote visual inspection apparatus of claim 16, wherein the light housing is provided by a control and display module.

22. The remote visual inspection apparatus of claim 16, wherein the plurality of optical fibers simultaneously receive the narrow band laser light from the laser diode and simultaneously emit the narrow band laser light at the camera head assembly.

23. The remote visual inspection apparatus of claim 10, wherein said first wavelength converter is disposed in said camera.

24. The remote visual inspection apparatus of claim 23, wherein said detachable tip comprises a first window adjacent the first wavelength converter and a second window adjacent the second wavelength converter, the first white light passing through the first window to illuminate the target area and the second white light passing through the second window.

25. The remote visual inspection apparatus of claim 10, wherein said detachable tip comprises a waveguide for receiving the second portion of said narrow band laser light emitted from the distal end of the second portion of said fiber optic bundle and directing it to the second wavelength converter.

26. The remote visual inspection apparatus of claim 16, further comprising a second optical diffuser disposed only in said camera head assembly for receiving said narrow band laser light emitted from said fiber optic bundle, said second optical diffuser for receiving the emitted narrow band laser light and for converting the narrow band laser light to white light.

27. The remote visual inspection apparatus of claim 19, wherein the second optical diffuser is disposed in the detachable tip.
